(19) United States
(12) Patent Application Publication
(10) Pub. No.: US 2021/0106716 A1
(43) Pub. Date: Apr. 15, 2021

(54) BIOFRAGMENTABLE HEMOSTATIC SPONGE

(71) Applicant: TAIPEI MEDICAL UNIVERSITY, TAIPEI CITY (TW)

(72) Inventors: Jen-Chang YANG, TAIPEI CITY (TW); Liang-Yu CHANG, TAIPEI CITY (TW); Shih-Han HUNG, TAIPEI CITY (TW); Hsin-Jen CHANG, TAIPEI CITY (TW); Nai-Chia TENG, TAIPEI CITY (TW)

(21) Appl. No.: 16/597,289

(22) Filed: Oct. 9, 2019

(51) Int. Cl. A61L 15/22; A61L 15/42; A61F 13/20; A61L 15/64

(52) U.S. Cl. CPC A61L 15/225; A61L 15/425; A61L 2400/04; A61L 15/64; A61F 13/2005

(57) ABSTRACT

The present disclosure provides a biofragmentable sponge, comprising a three-dimensional porous scaffold formed by freeze-drying a mixture of about 5% to about 20% (w/w) of silk protein, about 5% to about 85% (w/w) of a water soluble synthetic polymer or a mixture of water soluble synthetic polymers, and about 10% to about 90% (w/w) of a polysaccharide.

### FIELD OF THE INVENTION

The present disclosure relates to products having biofragmentable hemostatic characteristics. More particularly, the invention relates to a sponge having silk protein.

### BACKGROUND OF THE INVENTION

Hemostasis is the physiological process that stops bleeding at the site of an injury while maintaining normal blood flow elsewhere in the circulation, a complex physiological process involving cells (platelets, especially but also fibroblasts), and soluble (coagulation factors and inhibitors) and insoluble proteins (extracellular matrix proteins). Materials have been developed for controlling bleeding in situations where conventional aid is unavailable or less than optimally effective. Exemplary known dressings include microfibrillar collagen hemostat, chitosan hemostats, zeolites, thrombin and fibrin glue products and foam-forming agents etc.

US2012/0,114,592A1 provides a biodegradable hemostatic foam comprising a polymer blend of a water-soluble polymer and a phase-separated polyurethane comprising an amorphous segment and a crystalline segment. U.S. Pat. No. 10,039,721B2 provides a dressing including a silk protein layer and a hydrophilic glycoside compound, and the hydrophilic glycoside compound is coated on the silk protein layer.

However, there is an ongoing need to improve the capability to readily promote hemostasis.

### SUMMARY OF THE INVENTION

The present disclosure provides a biofragmentable sponge, comprising a three-dimensional porous scaffold, which is formed by freeze-drying a solution of about 5% to about 20% (w/w) of silk protein, about 5% to about 85% (w/w) of a water soluble polymer or a mixture of water soluble synthetic polymers, and about 10% to about 90% (w/w) of a polysaccharide or a mixture of polysaccharides.

The present disclosure also provides a method of preparing a biofragmentable sponge, comprising mixing about 5% to about 20% (w/w) of silk protein, about 5% to about 85% (w/w) of a water soluble synthetic polymer or a mixture of water soluble synthetic polymers, and about 10% to about 90% (w/w) of a polysaccharide or a mixture of polysaccharides to form a solution, and then freeze-drying the solution to form the biofragmentable sponge.

In one embodiment, before the freeze-drying, the solution is placed under a reduced temperature. In one embodiment, the temperature is reduced in a 2-stage manner. In a further embodiment, the temperature at the first stage is reduced to about 4+/−about 2° C. and the temperature at the second stage is gradually reduced to about −20+/−about 2° C. at a rate of greater than 5° C./min. In a further embodiment, the solution is then placed at about −20+/−about 2° C. for more than 14 hours.

In some embodiments, the silk protein is silkworm silk protein or spider silk protein or a fragment thereof. In one embodiment, the silk protein is in the form of a lyophilized form or structure. In some embodiments, the silk protein is in an amount ranging from about 5% to about 15% (w/w), about 5% to about 10% (w/w), about 8% to about 20% (w/w), about 10% to about 20% (w/w), about 12% to about 20% (w/w), about 15% to about 20% (w/w) or about 8% to about 20% (w/w).

In some embodiments, the water soluble synthetic polymer include, but are not limited to, polyethylene glycol (PEG), polyvinyl pyrrolidone (PVP), polyvinyl alcohol (PVA), polyethylene oxide (PEO), poly(propylene oxide) (PPO), poly(propylene glycol) (PPG) and a mixture thereof. In some embodiments, the polymer is in an amount ranging from about 5% to about 80% (w/w), about 5% to about 75% (w/w), about 5% to about 70% (w/w), about 5% to about 65% (w/w), about 5% to about 60% (w/w), about 5% to about 55% (w/w), about 5% to about 50% (w/w), about 5% to about 45% (w/w), about 5% to about 40% (w/w), about 5% to about 35% (w/w), about 5% to about 30% (w/w), about 5% to about 25% (w/w), about 5% to about 20% (w/w), about 5% to about 15% (w/w), about 10% to about 85% (w/w), about 15% to about 85% (w/w), about 20% to about 85% (w/w), about 25% to about 85% (w/w), about 30% to about 85% (w/w), about 35% to about 85% (w/w), about 40% to about 85% (w/w), about 45% to about 85% (w/w), about 50% to about 85% (w/w), about 55% to about 85% (w/w), about 60% to about 85% (w/w), about 65% to about 85% (w/w), about 70% to about 85% (w/w) or about 75% to about 85% (w/w).

In some embodiments, the polysaccharide includes, but are not limited to, cellulose, cellulose derivatives, methylcellulose (MC), carboxymethylcellulose sodium, carboxymethylcellulose (CMC), ethyl (hydroxyethyl) cellulose (EHEC), ethylcellulose, hydroxypropyl cellulose, hydroxypropyl methylcellulose (HPMC), ethylcellulose, alkyl cellulose, alkoxy cellulose, hydroxy ethyl cellulose, chitosan, chitosan hydrolysate and glycogen. In some embodiments, the polysaccharide is in an amount ranging from about 10% to about 85% (w/w), about 10% to about 80% (w/w), about 10% to about 75% (w/w), about 10% to about 70% (w/w), about 10% to about 65% (w/w), about 10% to about 60% (w/w), about 10% to about 55% (w/w), about 10% to about 50% (w/w), about 10% to about 45% (w/w), about 10% to about 40% (w/w), about 10% to about 35% (w/w), about 10% to about 30% (w/w), about 10% to about 25% (w/w), about 10% to about 20% (w/w), about 20% to about 90% (w/w), about 25% to about 90% (w/w), about 30% to about 90% (w/w), about 35% to about 90% (w/w), about 40% to about 90% (w/w), about 45% to about 90% (w/w), about 50% to about 90% (w/w), about 55% to about 90% (w/w), about 60% to about 90% (w/w), about 65% to about 90% (w/w), about 70% to about 90% (w/w), about 75% to about 90% (w/w) or about 80% to about 90% (w/w).

The present disclosure also provides a dressing, comprising a biofragmentable porous sponge of the present disclosure. In some embodiments, the dressing is a nasal pack, a porous scaffold, hemostatic sponge, scaffold, or substance delivering implant. In some embodiments, the nasal packing is in a form of a plug, sheet or tampon.

The present disclosure also provides a method for controlling bleeding, improving wound healing or closure, preventing tissue adhesion packing antrum or cavity of a body or supporting tissue regeneration, comprising applying the biofragmentable porous sponge of the present disclosure to a site in need thereof. In some embodiments, the method of the present disclosure maintains wound stabilization for 24 hours and gradually degrades after 72 hours. In a further embodiment, the porous sponge of the present disclosure can maintain 30 days before degradation.

### BRIEF DESCRIPTION OF THE DRAWING

FIG. 1 shows the photos of the lumps of RhinoSilk I, RhinoSilk II and NasoPore® soaked in normal saline at 0 hour, 24 hours, 48 hours, 72 hours and 96 hours.

FIG. 2 shows the compression strengths of RhinoSilk I and NasoPore®.

FIG. 3 shows the structures of RhinoSilk I and NasoPore® under electron microscope.

### DETAILED DESCRIPTION OF THE INVENTION

Various terms are used herein consistent with their meanings as understood by those having ordinary skill in the art. By way of further illustration, several terms are defined as follows.

Unless otherwise clear from context, the term “a” may be understood to mean “at least one.” As used in this application, the term “or” may be understood to mean “and/or.”

As used in this application, the terms “about” and “approximately” are used as equivalents. Any numerals used in this application with or without “about/approximately” are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the terms “silk protein” and “silk polypeptide” refer to a fibrous protein/polypeptide that can be used to produce a silk fiber, and/or a fibrous protein complex.

As used herein, the term “biofragmentable” refers to materials that, when introduced into cells, are broken down into components that cells can either reuse or dispose of without significant toxic effects on the cells.

As used herein, the term “porosity”: The term “porosity” as used herein, refers to a measure of void spaces in a material and is a fraction of volume of voids over the total volume, as a percentage between 0 and 100%.

The present invention provides a generally, porous and biofragmentable absorbent sponges for packing antrum or cavities in human or animal body. The sponges of the present disclosure can maintain wound stabilization for 24 hours and gradually degrade after 72 hours.

The biofragmentable sponges of the present disclosure are biofragmentable, having the ability of a polymer to degrade and disintegrate into chemical or biochemical products. Further, the sponges have bioresorbable feature, so it can be metabolized by the human or animal body and internally used in the body.

The biofragmentable sponge comprises a three-dimensional porous scaffold which is particularly suitable for packing antra or cavities of the human or animal body with an ability to stop bleeding and preserve tissue.

The biofragmentable sponge of the present disclosure is prepared by freeze-drying the solution of about 5% to about 20% (w/w) of silk protein, about 5% to about 85% (w/w) of a water soluble synthetic polymer or a mixture of water soluble synthetic polymers, and about 10% to about 90% (w/w) of a polysaccharide or a mixture of polysaccharides.

The solution of silk protein, the water-soluble synthetic polymer or a mixture thereof and the polysaccharide or mixture thereof is placed under a reduced temperature in a 2-stage manner before the freeze-drying. The temperature at the first stage is reduced to about 4+/−about 2° C. so that the temperature of the solution reaches to about 4+/−about 2° C. Then, the temperature of the solution is gradually reduced to about −20+/−about 2° C. at a rate of greater than 5° C./min. Furthermore, the solution is then placed at about −20+/−about 2° C. for more than 14 hours.

Accordingly, the present disclosure provides a fragmentable sponge with high porosity and pores are interconnected. The sponge also can be used as a carrier for other therapeutics/bioactive molecules/cells (primary or stem cells) towards tissue engineering and other biomaterial applications.

The sponge of the present disclosure can be used for various applications; for example, absorbent foam dressing for exudating wounds, dressing for nasal interventions, ear and other body cavities, as a drug and cell carrier and cell growth matrix, as carrier for various therapeutic and antimicrobial agents or as a cover for compromised tissues.

Those skilled in the art will recognize that the polyelectrolyte complex gel, soft tissue augmentation implant and methods of the present invention will have various other uses in addition to the above described embodiments. They will appreciate that the foregoing specification and accompanying drawings are set forth by way of illustration and not limitation of the invention. It will further be appreciated that various modifications and changes may be made therein without departing from the spirit and scope of the present invention, which is to be limited solely by the scope of the appended claims.

### EXAMPLES

### Example 1 Preparation of the Biofragmentable Sponge of the Present Disclosure

The following ratios of poly(vinyl alcohol)/poly(vinylpyrrolidinone), agarose and carboxylmethyl chitosan (Table 1) were mixed. The resulting mixtures were filled into a mold for forming under a temperature of 4±2° C. After the temperature of the solution reached to 4±2° C., the temperature of the solution was gradually reduced to −20+/−2° C. at a rate of greater than 5° C./min and then stood at −20+/−2° C. for more than 14 hours to form sponges. The resulting sponges were freeze-dried to obtain a dried sponge.

| | | | | carboxyl- |
|---|---|---|---|---|
| | | Poly(vinyl alcohol)/ | | methyl |
| | Silk | poly(vinylpyrrolidinone) | Agarose | chitosan |
| | protein | (w/w) | (w/w) | (w/w) |
| Formulation A | 5% | 5% | 60% | 30% |
| Formulation B | 5% | 85% | 5% | 5% |
| Formulation C | 20% | 5% | 50% | 25% |
| Formulation D | 10% | 50% | 10% | 30% |
| Formulation F | 20% | 70% | 0% | 10% |
| (RhinoSilk II) | | | | |
| Formulation G | 15% | 20% | 50% | 15% |
| Formulation H | 5% | 5% | 65% | 25% |
| (RhinoSilk I) | | | | |
| Formulation I | 5% | 15% | 50% | 30% |

### Example 2 Degradation Testing for the Biofragmentable Sponge of the Present Disclosure

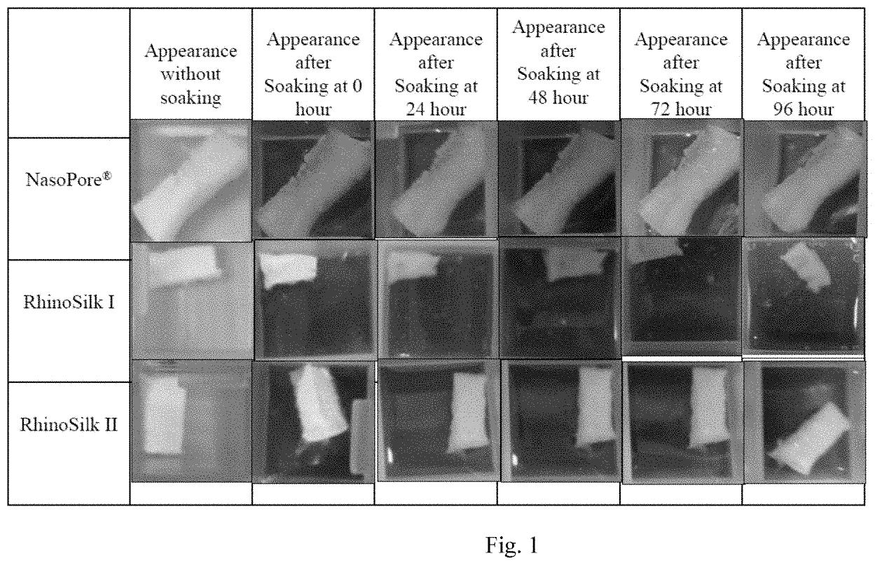

The sponge obtained from Formulations H and G of Example 1 (RhinoSilk I and RhinoSilk II) and commercial product NasoPore® were cut to lumps with a size of 10×5×5 mm³. The lumps of RhinoSilk and NasoPore® were placed to containers each with 3 ml normal saline, respectively and placed at 37+/−2° C. The lumps were taken photos at certain time intervals (see FIG. 1) and subjected to disintegration test after 96 hours using a strength of 100 g to determine the biofragmentability of the lumps (TA.XT Plus Texture Analyser, Stable Micro Systems, United Kingdom).

### Example 3 Anti-Compression Testing for the Biofragmentable Sponge of the Present Disclosure

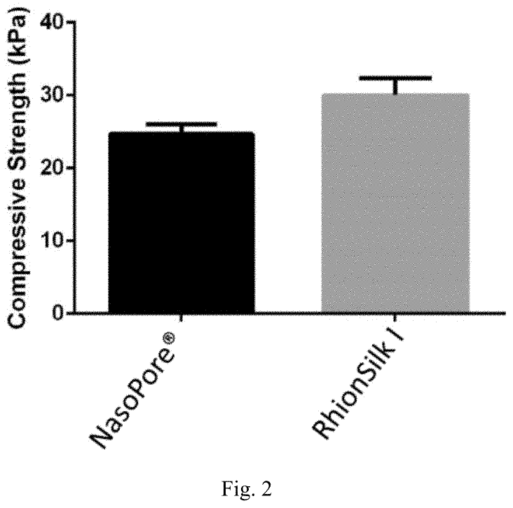

The sponge obtained from Formulation H of Example 1 (RhinoSilk) and commercial product NasoPore® were cut to lumps with a size of 10×5×5 mm³. The compression strength of the lumps of RhinoSilk and NasoPore® were measured by Texture Profile Analysis (TPA) (TA.XT Plus Texture Analyser, Stable Micro Systems, United Kingdom). FIG. 2 shows that the compression strength of RhinoSilk is superior than that of NasoPore®.

### Example 4 Comparison on Structures of the Biofragmentable Sponge of the Present Disclosure

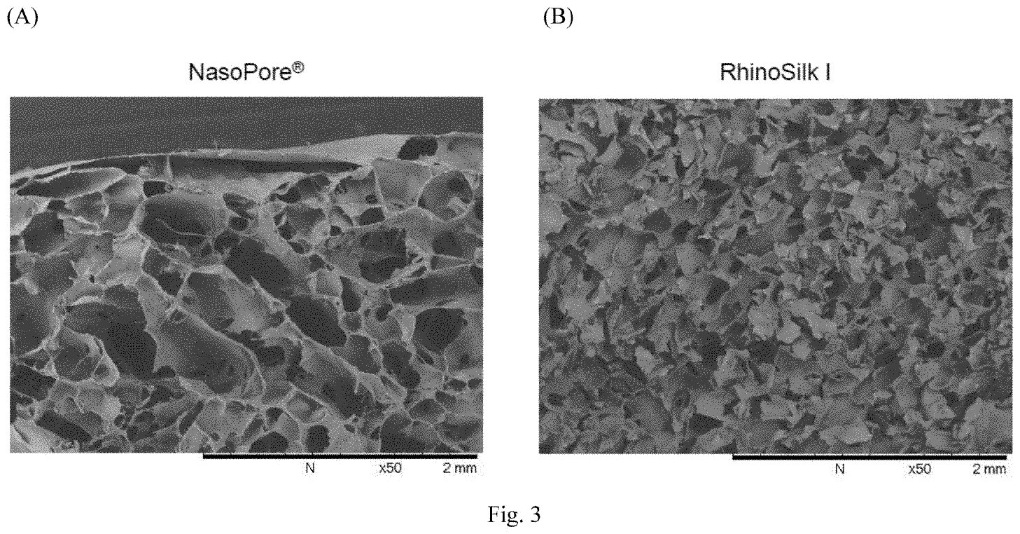

The sponge obtained from Formulation H of Example 1 (RhinoSilk) and commercial product NasoPore® were cut to lumps with a size of 10×5×5 mm³. The lumps of RhinoSilk and NasoPore® were observed using electron microscopy (TM-3030, Hitachi, Japan). The structures of RhinoSilk and NasoPore® were shown in FIG. 3.

What is claimed is:

1. A biofragmentable sponge, comprising a three-dimensional porous scaffold, which is formed by freeze-drying a solution of about 5% to about 20% (w/w) of silk protein, about 5% to about 85% (w/w) of a water soluble synthetic polymer or a mixture of water soluble synthetic polymers, and about 10% to about 90% (w/w) of a polysaccharide or a mixture of polysaccharides.

2. The biofragmentable porous sponge of claim 1, wherein the silk protein is silkworm silk protein or spider silk protein or a fragment thereof.

3. The biofragmentable porous sponge of claim 1, wherein the silk protein is in the form of a lyophilized form or structure.

4. The biofragmentable porous sponge of claim 1, wherein the silk protein is in an amount ranging from about 5% to about 15% (w/w).

5. The biofragmentable porous sponge of claim 1, wherein the water soluble synthetic polymer is a polyethylene glycol, polyvinyl pyrrolidone, polyvinyl alcohol, polyethylene oxide, poly(propylene oxide), poly(propylene glycol) or a mixture thereof.

6. The biofragmentable porous sponge of claim 1, wherein the polymer is in an amount ranging from about 5% to about 80% (w/w).

7. The biofragmentable porous sponge of claim 1, wherein the polysaccharide is cellulose, cellulose derivatives, methylcellulose, carboxymethylcellulose sodium, carboxymethylcellulose (CMC), ethyl (hydroxyethyl) cellulose (EHEC), ethylcellulose, hydroxypropyl cellulose, hydroxypropyl methylcellulose (HPMC), ethylcellulose, alkyl cellulose, alkoxy cellulose, hydroxy ethyl cellulose, chitosan, chitosan hydrolysate or glycogen.

8. The biofragmentable porous sponge of claim 1, wherein the polysaccharide is in an amount ranging from about 10% to about 85% (w/w).

9. The biofragmentable porous sponge of claim 1, wherein before the freeze-drying, the solution is placed under a reduced temperature in a 2-stage manner.

10. The biofragmentable porous sponge of claim 9, wherein the temperature at the first stage is reduced to about 4+/−about 2° C. and the temperature at the second stage is gradually reduced to about −20+/−about 2° C. at a rate of greater than 5° C./min.

11. The biofragmentable porous sponge of claim 10, wherein the solution is then placed at about −20+/−about 2° C. for more than 14 hours.

12. A method of preparing a biofragmentable sponge, comprising mixing about 5% to about 20% (w/w) of silk protein, about 5% to about 85% (w/w) of a water soluble synthetic polymer or a mixture of water soluble synthetic polymers, and about 10% to about 90% (w/w) of a polysaccharide or a mixture of polysaccharides to form a solution, and then freeze-drying the solution to form the biofragmentable sponge.

13. The method of claim 12, before the freeze-drying, further comprising a step of placing the solution at a reduced temperature in a 2-stage manner.

14. The method of claim 13, wherein the temperature at the first stage is reduced to about 4±about 2° C. and the temperature at the second stage is gradually reduced to about −20+/−about 2° C. at a rate of greater than 5° C./min.

15. The method of claim 14, wherein the solution is then placed at about −20+/−about 2° C. for more than 14 hours.

16. A dressing, comprises a biofragmentable porous sponge of claim 1.

17. The dressing of claim 16, wherein the dressing is a nasal pack, a porous scaffold, hemostatic sponge or substance delivering implant.

18. The dressing of claim 17, wherein the nasal packing is in a form of a plug, sheet or tampon.

19. A method for controlling bleeding, improving wound healing or closure, preventing tissue adhesion packing antrum or cavity of a body or supporting tissue regeneration, comprising applying the biofragmentable porous sponge of claim 1 to a site in need thereof.

20. The method of claim 19, wherein the method maintains wound stabilization for 24 hours and gradually degrades after 72 hours.
